(19) United States
(12) Patent Application Publication
(10) Pub. No.: US 2020/0155503 A1
(43) Pub. Date: May 21, 2020

(54) COMPOSITIONS FOR USE FOR TREATING CUTANEOUS LEISHMANIASIS

(71) Applicant: BIONOOX SA, Epalinges (CH)

(72) Inventor: Edward STARCKMANN, Lausanne (CH)

(73) Assignee: BIONOOX SA, Epalinges (CH)

(21) Appl. No.: 16/634,781

(22) PCT Filed: Aug. 3, 2018
(86) PCT No.: PCT/EP2018/071168

§ 371 Date: Jan. 28, 2020

(30) Foreign Application Priority Data
Aug. 3, 2017 (EP) 17306038.5

(51) Int. Cl. A61K 31/355; A61K 31/245; A61P 33/02; A61K 31/045

(52) U.S. Cl. CPC A61K 31/355; A61K 31/045; A61P 33/02; A61K 31/245

(57) ABSTRACT

The invention relates to the use of a composition comprising dihydroquercetin, alpha-tocopherol, and optionally bisabolol and/or a compound of formula (I) for use in the treatment of leishmaniasis, especially cutaneous leishmaniasis. The invention further relates to the use of such a composition for treating inflammation induced by leishmaniasis, especially cutaneous leishmaniasis. The invention also relates to a method for treating leishmaniasis, especially cutaneous leishmaniasis, and/or the associated inflammation by administering an effective amount of a composition comprising dihydroquercetin, bisabolol, alpha-tocopherol and optionally bisabolol and/or a compound of formula (I) to a subject in need thereof.

### FIELD OF INVENTION

The present invention pertains to the field of the treatment of leishmaniasis, especially cutaneous leishmaniasis.

In particular, the present invention relates to a composition comprising dihydroquercetin and alpha-tocopherol, for use in the treatment of leishmaniasis, especially in topical treatment of cutaneous leishmaniasis. The compositions of the invention may further comprise bisabolol and/or a compound of formula (I) such as for example 3-(2-chloroacetamido) ethyl benzoate or 4-(2-chloroacetamido) ethyl benzoate.

This invention also relates to a method for treating a subject in need thereof, infected by a leishmaniasis parasite, said method comprising administering to said subject a composition comprising dihydroquercetin and alpha-tocopherol.

### BACKGROUND OF INVENTION

Leishmaniasis is a parasitic disease spread by the bite of infected sandflies. It is endemic in 88 countries throughout Africa, Asia, Europe, and North and South America and there are an estimated 12 million cases worldwide, with 1.5 to 2 million new cases each year.

Leishmaniasis is divided into four main clinical forms and is caused by parasitic protozoa of the genus Leishmania. There are over 20 species and subspecies that infect humans via the bite of sandflies of subfamily phlebotominae. The life cycle of Leishmania parasites begins when an infected fly bites and injects Leishmania infective promastigotes which are present in its proboscis directly in the skin of a host. These promastigotes are then phagocytosed by macrophages prior to their transformation into amastigotes which are able to divide. Upon maximum levels of amastigote divisions, the macrophages burst release more amastigotes that are again re-phagocytosed. When an uninfected fly bites an infected individual, it ingests the amastigotes and these transform into promastigotes and divide in the midgut of the fly. Finally, these promastigotes migrate to the proboscis of the fly and makes it able to transmit the disease.

The clinical features of the disease depend on the causative species and can range from simple, self-healing skin sores as found in cutaneous leishmaniasis, to severe, life threatening diseases affecting the internal organs of the body such as visceral leishmaniasis.

One of the most common forms of the disease is cutaneous leishmaniasis that occurs most commonly in Iran, Afghanistan, Syria, Saudi Arabia, Peru and Brazil. Cutaneous leishmaniasis is characterized by skin lesions such as sores, which typically develop within several weeks or months after exposure. However, in some patients, the sores first appear months or years after the infection in a context of skin traumatism, e.g. skin wounds or surgery. The sores can change in size and appearance over time. They typically progress from small papules to nodular plaques, and often lead to open sores with a raised border and central crater (ulcer), which can be covered with scales or crust. The lesions are usually painless but can be painful, particularly if open sores become infected with bacteria. Satellite lesions, regional lymphadenopathy, and nodular lymphangitis can be noted. The sores may heal eventually, even without treatment. However, they can last for months or years and typically result in scarring.

A potential concern applies to some of the Leishmania species in South and Central America, because some parasites can spread from the skin to the mucosal surfaces of the nose or mouth and cause sores therein. This form of leishmaniasis, mucosal leishmaniasis, might not be noticed until years after the original skin sores appear to have healed. Although mucosal leishmaniasis is uncommon, it has occurred in travelers and expatriates whose cases of cutaneous leishmaniasis were not treated, or were inadequately treated. The initial clinical manifestations typically involve the nose such as chronic stuffiness, bleeding, and inflamed mucosa or sores, and less often the mouth. In advanced cases, ulcerative destruction of the nose, mouth, and pharynx can be noted, such as perforation of the nasal septum.

Anti-parasitic pentavalent antimonials, such as sodium stibogluconate or meglumine antimoniate, are the basis for all treatment of leishmaniasis. Long courses of these drugs are often required. Pentavalent antimony is thought to work by inhibition of adenosine triphosphate synthesis. The antimonial agent specifically used in the United States is sodium stibogluconate (Pentostam®). After 20 days of treatment with pentavalent antimonials, there is usually evidence of healing, but lesions may not be re-epithelialized completely. Healing is determined by a healed appearance at two months, no relapse at 12 months, and no subsequent mucosal disease. The response to a particular regimen may vary not only among Leishmania species but also for the same species in different geographic regions. Pentavalent antimonials have a high incidence of side effects. Side effects include aching; arthralgia; fatigue; gastrointestinal upset; elevation of amylase, lipase, and liver enzyme levels; leukopenia; anemia; and electrocardiographic abnormalities.

There is general agreement to consider that the commonly available drugs for the treatment of leishmaniasis have severe side effects, high cost and low efficacy (Shukla et al., Applied Biochemistry and Biotechnology, 2010, Vol. 160, pp. 2208-2218.). Accordingly, there is still a need for improved treatment of leishmaniasis, and more especially for treatment of cutaneous leishmaniasis.

The U.S. patent application Ser. No. 15/503,095 discloses haloacetamidobenzoic acid compounds having a significant antiparasitic effects. Nevertheless, such compounds present a non-negligible toxicity to the host organism, thereby hindering their therapeutic application.

To the Applicant's surprise, such haloacetamidobenzoic acid compounds present a synergistic effect with dihydroquercetin, alpha-tocopherol and/or bisabolol. Such synergy paves the way to reducing the effective concentration of the haloacetamidobenzoic acid compounds, thus maintaining their effectiveness while drastically reducing their toxicity.

Dihydroquercetin, alpha-tocopherol and bisabolol when evaluated individually, showed no or limited effect against Leishmania parasites.

The Applicant surprisingly found that a composition comprising dihydroquercetin, alpha-tocopherol, and bisabolol, provides a potent leishmaniasis treatment which reduces side effects and improves the comfort of the treated subject compared to prior art treatments.

Even more surprisingly, it was found in this invention that a composition comprising dihydroquercetin and alpha-tocopherol may present the same anti-parasitic effects as potent anti-parasitic agents such as for example the haloacetamidobenzoic acid compounds of the U.S. Ser. No. 15/503,095 patent application. The therapeutic action of the composition according to the invention may especially comprise the following effects:
killing the parasite, i.e. anti-leishmanial activity;
reducing the risk of resistance of the parasite to the treatment;
limitation of inflammation and/or local skin necrosis;
limitation of dissemination of the parasite to others tissues;
activation of the wound healing process; and/or
antibacterial and antifungal activity, thereby reducing the risk of secondary infection.

### SUMMARY

The Applicant has found that the association of natural products such as dihydroquercetin and alpha-tocopherol may yield to similar anti-leishmanial effects as synthetic anti-leishmanial agents known in the art.

In a first aspect, the present invention relates to a composition comprising dihydroquercetin and alpha-tocopherol for use in the treatment of leishmaniasis.

In the composition for use according to the invention the concentration of dihydroquercetin ranges from 2% to 8.0% w/w, preferably from 2.5% to 7.0% w/w and the concentration of alpha-tocopherol ranges from 0.1% to 3% w/w, preferably from 0.5% to 2% w/w, respectively.

The composition for use of the invention can further comprise bisabolol, in a concentration ranging from 0.1% to 8.0% w/w, preferably from 2.5% to 4.0% w/w.

To the Applicant's surprise, dihydroquercetin and/or alpha-tocopherol present a synergistic effect along with the haloacetamidobenzoic acid compounds of formula (I) against leishmania.

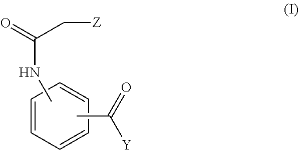

wherein: Z represents a halogen atom selected from the group consisting of Cl, Br, I and F; and Y represents a substituent selected from a group consisting of C₂H₅O—; —NH—CO—NH—R wherein R represents H, an alkyl or aryl group; and N—R1 wherein R1 is an alkyl or heterocyclic group.

Thus, in one embodiment, the composition for use further comprises a compound of formula (I). The compound of formula (I) can namely be 3-(2-chloroacetamido) ethyl benzoate or 4-(2-chloroacetamido) ethyl benzoate. In one embodiment, the concentration of the compound of formula (I) in the composition for use according to the invention ranges from 0.05% to 1.0% w/w, preferably from 0.1% to 0.6% w/w, in weight relative to the total composition.

The composition for use of the invention may adequately be formulated in association with at least one pharmaceutically and/or cosmetically acceptable vehicle such as triglycerides, animal fat, vegetable fat, higher alcohols, glycols, mineral oil, and a mixture thereof. It can further comprise at least one component selected from surfactants, pigments, stabilizers, emollients, humectants, perfumes, preservatives, and a mixture thereof. Such formulations can be in the form of a cream, a gel, an ointment, a solution, an emulsion, a mask, a milk, a lotion, a serum, a paste, a stick, a foam or a suspension; preferably a cream or a stick.

In one embodiment, the composition is for use in the treatment of cutaneous, mucosal or visceral leishmaniasis, preferably cutaneous leishmaniasis. In one embodiment, the parasitic protozoa responsible of leishmaniasis are Leishmania aethiopica, Leishmania amazonensis, Leishmania braziliensis, Leishmania donovani, Leishmania guyanensis, Leishmania infantum, Leishmania lainsoni, Leishmania lindenbergi, Leishmania mexicana, Leishmania major, Leishmania naiffi, Leishmania panamensis, Leishmania peruviana, Leishmania shawi, Leishmania tropica and/or Leishmania venezuelensis; preferably Leishmania amazonensis, Leishmania donovani, Leishmania mexicana and/or Leishmania major.

In other aspects, the present invention relates to a pharmaceutical and/or cosmetic composition as well as to a device, preferably a delivery device, that comprise the composition as previously described, for the treatment of leishmaniasis.

### Definitions

In the present invention, the following terms have the following meaning:
“about” preceding a figure means more or less 10% of the value of said figure.
“alkyl” refers to a hydrocarbyl radical of formula CₙH₂₊₁ wherein n is a number greater than or equal to 1. Generally, alkyl groups of this invention comprise from 1 to 12 carbon atoms, preferably from 1 to 6 carbon atoms, even more preferably 1 to 3 atoms. Alkyl groups may be linear or branched and may be substituted as indicated herein. Suitable alkyl groups include methyl, ethyl, propyl (n-propyl, i-propyl, n-butyl), butyl (i-butyl, s-butyl and t-butyl), pentyl and its isomers (e.g. n-pentyl, iso-pentyl), and hexyl and its isomers (e.g. n-hexyl, iso-hexyl). In one embodiment, the alkyl group designates an ethyl group.
“aryl” refers to a polyunsaturated, aromatic hydrocarbyl group having a single ring or multiple aromatic rings fused together (such as nathyl or linked covalently, typically containing 5 to 20, and preferably 6 to 12, carbon atoms having one or more aromatic rings among which it is possible to cite the phenyl group, the biphenyl group, the 1-naphthyl group, the 2-naphthyl group, the tetrahydronaphthyl group, the indanyl group and the binaphthyl group. In one embodiment, aryl designates a phenyl group. In one embodiment, the aryl group is substituted by at least one substituent selected from a group comprising, halogens or alkyl groups, —OH, —SH₂, NH₂, —COOH, methoxy, ethoxy and —CHO groups.
“companion cosmetic composition” and “add-on non-therapeutic composition” refers to a cosmetic composition intended to assist a patient in the management of a therapy-related cutaneous or mucosal discomfort. Advantageously, a companion cosmetic composition is safe, do not contain phototoxic and/or photosensitizing components and show no toxicity. Moreover, a companion cosmetic composition does not affect the effectiveness of the therapy.
“cosmetic composition” refers to a composition intended to be in contact with the diverse superficial parts of the human body, in particular the skin or any mucous membranes, in sight, exclusively or mainly, to clean them, to perfume them, to protect them, to maintain them in good condition, to modify their aspect or to correct the body odors. In one embodiment, a cosmetic composition of the invention aims at reducing or preventing the appearance of the visible cutaneous or mucosal signs of leishmaniasis, thereby maintaining the skin or mucosa in good condition and/or or modify their aspects.
“cosmetically acceptable” refers to a component that is suitable for use in contact with the skin or any mucous membranes without inducing undue adverse side effects (such as toxicity, irritation, allergic response, and the like).
“cosmetically acceptable base” refers to a cosmetically acceptable vehicle comprising a lipophilic component.
“infected areas” refers to any part of the body where inflammation caused by leishmaniasis is present. Examples of such infected areas are, without limitation, skin and mucosa.
“pharmaceutic composition” refers to a composition comprising an active principle in association with a pharmaceutically acceptable vehicle. A pharmaceutical composition is for therapeutic use, and relates to health. In one embodiment, a pharmaceutical composition may be indicated for treating leishmaniasis.
“pharmaceutically acceptable” refers to a component that does not produce an adverse, allergic or other untoward reaction when administered to an animal, preferably a human. It includes any and all solvents, dispersion media, coatings, antibacterial and antifungal agents, isotonic and absorption delaying agents and the like. For human administration, injected preparations should meet sterility, pyrogenicity, general safety and purity standards as required by regulatory offices, such as, for example, FDA Office or EMA.
“subject” refers to a mammal, preferably a human. In one embodiment, a subject may be a “patient”, i.e. a warm-blooded animal, more preferably a human, who/which is awaiting the receipt of, or is receiving medical care or was/is/will be the object of a medical procedure, or is monitored for the development of leishmaniasis. In one embodiment, the subject is an adult (for example a subject above the age of 18). In another embodiment, the subject is a child (for example a subject below the age of 18). In one embodiment, the subject is a male. In another embodiment, the subject is a female.
“treating” or “treatment” refers to both therapeutic treatment and prophylactic or preventative measures; wherein the object is to prevent or slow down (lessen) or alleviate leishmaniasis-related symptoms. Those in need of treatment include those already with leishmaniasis as well as those prone to have leishmaniasis or those in whom leishmaniasis is to be prevented. A subject or mammal is successfully “treated” for leishmaniasis if, after receiving a therapeutic amount of a composition for use according to the present invention, the patient shows observable and/or measurable reduction or absence of one or more of the following: reduction in the number of pathogenic cells; reduction in the percent of total cells that are pathogenic; and/or relief to some extent, of one or more of the symptoms associated with leishmaniasis (including, without limitation, skin or mucosal lesions such as open or closed sores); reduced morbidity and mortality, and improvement in quality of life issues. The above parameters are readily measurable by routine procedures familiar to a physician.
“therapeutically effective amount” means the level or amount of the composition of the invention that is aimed at, without causing significant negative or adverse side effects to the target, (1) delaying or preventing the onset of leishmaniasis; (2) slowing down or stopping the progression, aggravation, or deterioration of one or more symptoms of leishmaniasis; (3) leading to ameliorations of the symptoms of leishmaniasis or alleviating the symptoms of leishmaniasis; or (4) reducing the severity or incidence of leishmaniasis symptoms. A therapeutically effective amount may be administered prior to the onset of leishmaniasis, for a prophylactic or preventive action. Alternatively, or additionally, the therapeutically effective amount may be administered after initiation of leishmaniasis symptoms, for a therapeutic action or maintenance of a therapeutic action.
“therapeutic composition” refers to a composition having the capacity, when administered in a suitable amount, of slowing down or stopping the progression, aggravation, or deterioration of one or more symptoms of leishmaniasis or of alleviating the symptoms of leishmaniasis.
“vehicle” refers to a substance with which the component of interest is mixed or wherein the component of interest is dissolved. In an embodiment, the vehicle may be a cosmetically acceptable base.
“w/w” designates the concentration of a compound in a composition as a percentage of the compound's weight relative to the total weight of the composition comprising said ingredient.

### DETAILED DESCRIPTION

In a first aspect, this invention relates to a composition comprising dihydroquercetin and alpha-tocopherol for use in the treatment of leishmaniasis.

Dihydroquercetin (DHQ) is the common name of 3,3′,4′,5,7-pentahydroxyflavone dehydrate, also called 2-(3,4-dihydroxyphenyl)-3,5,7-trihydroxy-4H-1-benzopyran-4-one dehydrate, also known as taxifolin (CAS number [480-18-2]). In one embodiment, DHQ is extracted from a type of larch wood, preferably from Siberian larch. In an embodiment, DHQ powder contains at least 96% w/w of pure dihydroquercetin by weight of the DHQ powder and corresponds to the technical requirements and sanitary rules on the basis of analytical and microbiological reports.

In one embodiment, the concentration of DHQ in the composition ranges from 0.1% to 10.0% w/w (i.e. in weight, by weight of the total composition), preferably from 2% to 8.0% w/w, more preferably from 2.5% to 7.0% w/w.

Alpha-tocopherol, commonly named vitamin E, is (2R)-2,5,7,8-Tetramethyl-2-[(4R,8R)-(4,8,12-trimethyltridecyl)]-6-chromanol (CAS number [59-02-9]).

In one embodiment, the concentration of alpha-tocopherol in the composition ranges from 0.05% to 5% w/w (i.e. in weight, by weight of the total composition), preferably from 0.1% to 3% w/w, more preferably from 0.5% to 2% w/w. In one embodiment, the composition for use according to the invention further comprises at least one compound selected from a list consisting of a compound of formula (I) and bisabolol.

In one embodiment, the composition for use according to the invention comprises dihydroquercetin, alpha-tocopherol and at least one compound selected from a list consisting of a compound of formula (I) and bisabolol.

Bisabolol is the common name of 6-methyl-2-(4-methyl-3-cyclohexen-1-yl)-5-hepten-2-ol, or 1-methyl-4-(1,5-dimethyl-1-hydroxyhex-4(5)-nyl)cyclohexen-1, also known as levomenol (CAS number [23089-26-1]). Bisabolol is a sesquiterpene found in various plants, including herbal tea and chamomile.

In one embodiment, the concentration of bisabolol in the composition ranges from 0.01% to 10.0% w/w (i.e. in weight, by weight of the total composition), preferably ranging from 0.1% to 8.0% w/w, more preferably from 1.0% to 4% w/w.

Compounds of formula (I) are described in detail in the U.S. patent application Ser. No. 15/503,095.

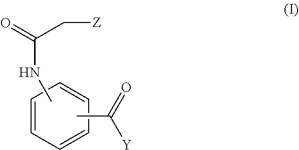

wherein:

Z represents a halogen atom selected from the group consisting of Cl, Br, I and F,

Y represents a substituent selected from a group consisting of C₂H₅O—, —NH—CO—NH—R wherein R represents H, an alkyl or aryl group and N—R1 wherein R1 is an alkyl or heterocyclic group.

Thus formula (I) comprises:
the ortho—compounds of formula I, being represented by the formula Ia, wherein Z and Y are as previously described.

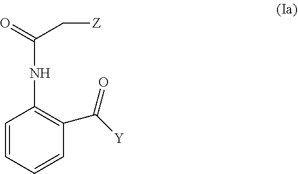

the meta—compounds of formula I, being represented by the formula Ib, wherein Z and Y are as previously described.

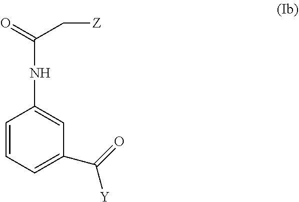

the para—compounds of formula I, being represented by the formula Ic, wherein Z and Y are as previously described.

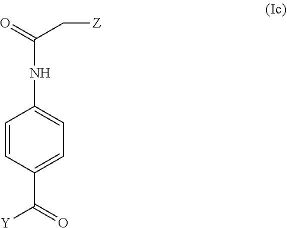

In one embodiment, the composition for use according to the invention comprises dihydroquercetin, alpha-tocopherol and at least one compound selected from a list consisting of a compound of formula (Ia) and bisabolol.

In one embodiment, the composition for use according to the invention comprises dihydroquercetin, alpha-tocopherol and at least one compound selected from a list consisting of a compound of formula (Ib) and bisabolol.

In one embodiment, the composition for use according to the invention comprises dihydroquercetin, alpha-tocopherol and at least one compound selected from a list consisting of a compound of formula (Ic) and bisabolol.

In one embodiment, the composition for use according to the invention comprises dihydroquercetin, alpha-tocopherol and at least one compound selected from a list consisting bisabolol and a compound of formula (I), wherein
—Z represents Cl or Br; and
Y represents C₂H₅O or —NH—CO—NH—CO—NH₂.

In one embodiment, the composition for use according to the invention comprises dihydroquercetin, alpha-tocopherol and at least one compound selected from a list consisting bisabolol and a compound of formula (Ia), wherein
—Z represents Cl or Br; and
Y represents C₂H₅O or —NH—CO—NH—CO—NH₂.

In one embodiment, the composition for use according to the invention comprises dihydroquercetin, alpha-tocopherol and at least one compound selected from a list consisting bisabolol and a compound of formula (Ib), wherein
—Z represents Cl or Br; and
Y represents C₂H₅O or —NH—CO—NH—CO—NH₂.

In one embodiment, the composition for use according to the invention comprises dihydroquercetin, alpha-tocopherol and at least one compound selected from a list consisting bisabolol and a compound of formula (Ic), wherein
—Z represents Cl or Br; and
Y represents C₂H₅O or —NH—CO—NH—CO—NH₂.

In one embodiment, the composition for use according to the invention comprises dihydroquercetin, alpha-tocopherol and at least one compound selected from a list consisting bisabolol and a compound of formula (Ib) or (Ic), wherein
—Z represents Cl or Br; and
Y represents C₂H₅O or —NH—CO—NH—CO—NH₂.

In one embodiment, the composition for use according to the invention comprises dihydroquercetin, alpha-tocopherol and at least one compound selected from a list consisting of a 3-(2-chloroacetamido) ethyl benzoate, 4-(2-chloroacetamido) ethyl benzoate and bisabolol.

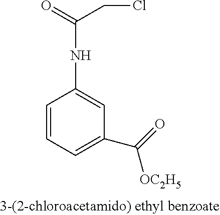 [SMILES: CCOC(=O)c1cccc(NC(=O)CCl)c1]

3-(2-chloroacetamido) ethyl benzoate is a compound of formula (I) also known as 3-(2 chloroacetylamino)benzoic acid ethyl ester, or ethyl 3 (chloroacetyl)aminobenzoate (CAS number [58915-19-8]).

4-(2-chloroacetamido) ethyl benzoate is a compound of formula (I), also known as ethyl 4-acetamidobenzoate, 4-(acetamino)-benzoic acid ethyl ester or N-Acetylbenzocaine (CAS number [5338-44-3]).

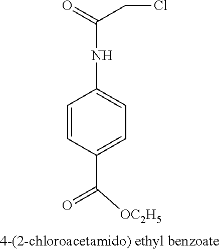 [SMILES: CCOC(=O)c1ccc(NC(=O)CCl)cc1]

In one embodiment, the concentration of the at least one compound of formula (I), formula (Ia), formula (Ib), formula (Ic), 3-(2-chloroacetamido) ethyl benzoate or 4-(2-chloroacetamido) ethyl benzoate in the composition ranges from 0.05% to 1.0% w/w, preferably from 0.1% to 0.6% w/w (i.e. in weight, by weight of the total composition).

In one embodiment, the concentration of the at least one compound of formula (I), formula (Ia), formula (Ib), formula (Ic), 3-(2-chloroacetamido) ethyl benzoate or 4-(2-chloroacetamido) ethyl benzoate in the composition ranges from 0.1% to 0.4% w/w. (i.e. in weight, by weight of the total composition).

In one embodiment, the composition for use according to the present invention comprises dihydroquercetin, alpha-tocopherol and bisabolol.

In one embodiment, the composition for use according to the present invention comprises dihydroquercetin, alpha-tocopherol, 3-(2-chloroacetamido) ethyl benzoate and bisabolol.

In one embodiment, the composition for use according to the present invention comprises dihydroquercetin, alpha-tocopherol, 4-(2-chloroacetamido) ethyl benzoate and bisabolol.

In one embodiment, the composition for use according to the present invention comprises dihydroquercetin and alpha-tocopherol, wherein the concentration of alpha-tocopherol in the composition ranges from 0.05% to 5% w/w (i.e. in weight, by weight of the total composition), preferably from 0.1% to 3% w/w, more preferably from 0.5% to 2% w/w.

In one embodiment, the relative ratio between DHQ and bisabolol in the composition (in weight by weight of the total composition), ranges from 0.01 to 1000, preferably from 0.25 to 80, more preferably from 0.625 to 7.

In one embodiment, the composition for use as previously described is in association with an acceptable vehicle. In one embodiment, the composition comprises DHQ, and alpha-tocopherol, in association with a cosmetically acceptable vehicle, especially a pharmaceutically or cosmetically acceptable base. In one embodiment, the composition comprises DHQ, alpha-tocopherol and optionally bisabolol, in association with a pharmaceutically acceptable vehicle. In one embodiment, the composition comprises DHQ, alpha-tocopherol and a compound of formula (I), in association with a pharmaceutically acceptable vehicle. In one embodiment, the composition comprises DHQ, alpha-tocopherol, a compound of formula (I) and bisabolol in association with a pharmaceutically acceptable vehicle. In one embodiment, the composition is a therapeutic composition for use in the treatment of leishmaniasis. In one embodiment, the composition is a pharmaceutical composition for use in the treatment of leishmaniasis. In one embodiment, the composition is a cosmetic composition.

In one embodiment, the composition comprising dihydroquercetin, alpha-tocopherol and optionally bisabolol is a companion cosmetic composition or an add-on non-therapeutic composition for use in a non-therapeutic method for alleviating the discomfort of cutaneous leishmaniosis symptoms.

In one embodiment, the composition comprises:
from 0.1% to 10.0% w/w, preferably from 2% to 8.0% w/w, more preferably from 2.5% to 7.0% w/w, of dihydroquercetin; and
from 0.05% to 5% w/w, preferably from 0.1% to 3% w/w, more preferably from 0.5% to 2% w/w, of alpha-tocopherol.

In one embodiment, the composition comprises:
from 0.1% to 10.0% w/w, preferably from 2% to 8.0% w/w, more preferably from 2.5% to 7.0% w/w, of dihydroquercetin;
from 0.05% to 5% w/w, preferably from 0.1% to 3% w/w, more preferably from 0.5% to 2% w/w, of alpha-tocopherol; and a pharmaceutically and/or cosmetically acceptable vehicle.

In one embodiment, the composition comprises:
from 0.1% to 10.0% w/w, preferably from 2% to 8.0% w/w, more preferably from 2.5% to 7.0% w/w, of dihydroquercetin;
from 0.05% to 5% w/w, preferably from 0.1% to 3% w/w, more preferably from 0.5% to 2% w/w, of alpha-tocopherol; and
from 0.01% to 10.0% w/w, preferably from 0.1% to 8.0% w/w, more preferably from 1% to 4% w/w, of bisabolol;

In one embodiment, the composition comprises:
from 0.1% to 10.0% w/w, preferably from 2% to 8.0% w/w, more preferably from 2.5% to 7.0% w/w, of dihydroquercetin;
from 0.05% to 5% w/w, preferably from 0.1% to 3% w/w, more preferably from 0.5% to 2% w/w, of alpha-tocopherol;
from 0.01% to 10.0% w/w, preferably from 0.1% to 8.0% w/w, more preferably from 1% to 4% w/w, of bisabolol; and
a pharmaceutically and/or cosmetically acceptable vehicle.

In one embodiment, the composition comprises:
from 0.1% to 10.0% w/w, preferably from 2% to 8.0% w/w, more preferably from 2.5% to 7.0% w/w, of dihydroquercetin;
from 0.05% to 5% w/w, preferably from 0.1% to 3% w/w, more preferably from 0.5% to 2% w/w, of alpha-tocopherol; and
from 0.01% to 1.0% w/w, preferably from 0.1% to 1.0% w/w, more preferably from 0.1% to 0.6% w/w, of a compound of formula (I) as previously described.

In one embodiment, the composition comprises:
from 0.1% to 10.0% w/w, preferably from 2% to 8.0% w/w, more preferably from 2.5% to 7.0% w/w, of dihydroquercetin;
from 0.05% to 5% w/w, preferably from 0.1% to 3% w/w, more preferably from 0.5% to 2% w/w, of alpha-tocopherol;
from 0.01% to 1.0% w/w, preferably from 0.1% to 1.0% w/w, more preferably from 0.1% to 0.6% w/w, of a compound of formula (I) as previously described; and
   a pharmaceutically and/or cosmetically acceptable vehicle.

In one embodiment, the composition comprises:
from 0.1% to 10.0% w/w, preferably from 2% to 8.0% w/w, more preferably from 2.5% to 7.0% w/w, of dihydroquercetin;
from 0.05% to 5% w/w, preferably from 0.1% to 3% w/w, more preferably from 0.5% to 2% w/w, of alpha-tocopherol; and
from 0.01% to 1.0% w/w, preferably from 0.1% to 1.0% w/w, more preferably from 0.1% to 0.6% w/w, of a compound of formula (Ia), (Ib) or (Ic) as previously described.

In one embodiment, the composition comprises:
from 0.1% to 10.0% w/w, preferably from 2% to 8.0% w/w, more preferably from 2.5% to 7.0% w/w, of dihydroquercetin;
from 0.05% to 5% w/w, preferably from 0.1% to 3% w/w, more preferably from 0.5% to 2% w/w, of alpha-tocopherol;
from 0.01% to 1.0% w/w, preferably from 0.1% to 1.0% w/w, more preferably from 0.1% to 0.6% w/w, of a compound of formula (Ia), (Ib) or (Ic) as previously described; and
   a pharmaceutically and/or cosmetically acceptable vehicle.

In one embodiment, the composition comprises:
from 0.1% to 10.0% w/w, preferably from 2% to 8.0% w/w, more preferably from 2.5% to 7.0% w/w, of dihydroquercetin;
from 0.05% to 5% w/w, preferably from 0.1% to 3% w/w, more preferably from 0.5% to 2% w/w, of alpha-tocopherol; and
from 0.01% to 1.0% w/w, preferably from 0.1% to 1.0% w/w, more preferably from 0.1% to 0.6% w/w, of a compound of formula (Ib) or (Ic) as previously described.

In one embodiment, the composition comprises:
from 0.1% to 10.0% w/w, preferably from 2% to 8.0% w/w, more preferably from 2.5% to 7.0% w/w, of dihydroquercetin;
from 0.05% to 5% w/w, preferably from 0.1% to 3% w/w, more preferably from 0.5% to 2% w/w, of alpha-tocopherol;
from 0.01% to 1.0% w/w, preferably from 0.1% to 1.0% w/w, more preferably from 0.1% to 0.6% w/w, of a compound of formula (Ib) or (Ic) as previously described; and
   a pharmaceutically and/or cosmetically acceptable vehicle.

In one embodiment, the composition comprises:
from 0.1% to 10.0% w/w, preferably from 2% to 8.0% w/w, more preferably from 2.5% to 7.0% w/w, of dihydroquercetin;
from 0.05% to 5% w/w, preferably from 0.1% to 3% w/w, more preferably from 0.5% to 2% w/w, of alpha-tocopherol; and
from 0.01% to 1.0% w/w, preferably from 0.1% to 1.0% w/w, more preferably from 0.1% to 0.6% w/w, of 3-(2-chloroacetamido) ethyl benzoate.

In one embodiment, the composition comprises:
from 0.1% to 10.0% w/w, preferably from 2% to 8.0% w/w, more preferably from 2.5% to 7.0% w/w, of dihydroquercetin;
from 0.05% to 5% w/w, preferably from 0.1% to 3% w/w, more preferably from 0.5% to 2% w/w, of alpha-tocopherol;
from 0.01% to 1.0% w/w, preferably from 0.1% to 1.0% w/w, more preferably from 0.1% to 0.6% w/w, of 3-(2-chloroacetamido) ethyl benzoate; and
   a pharmaceutically and/or cosmetically acceptable vehicle.

In one embodiment, the composition comprises:
from 0.1% to 10.0% w/w, preferably from 2% to 8.0% w/w, more preferably from 2.5% to 7.0% w/w, of dihydroquercetin;
from 0.05% to 5% w/w, preferably from 0.1% to 3% w/w, more preferably from 0.5% to 2% w/w, of alpha-tocopherol; and
from 0.01% to 1.0% w/w, preferably from 0.1% to 1.0% w/w, more preferably from 0.1% to 0.6% w/w, of 4-(2-chloroacetamido) ethyl benzoate.

In one embodiment, the composition comprises:
from 0.1% to 10.0% w/w, preferably from 2% to 8.0% w/w, more preferably from 2.5% to 7.0% w/w, of dihydroquercetin;
from 0.05% to 5% w/w, preferably from 0.1% to 3% w/w, more preferably from 0.5% to 2% w/w, of alpha-tocopherol;
from 0.01% to 1.0% w/w, preferably from 0.1% to 1.0% w/w, more preferably from 0.1% to 0.6% w/w, of 4-(2-chloroacetamido) ethyl benzoate; and
   a pharmaceutically and/or cosmetically acceptable vehicle.

In one embodiment, the composition comprises:
from 0.1% to 10.0% w/w, preferably from 2% to 8.0% w/w, more preferably from 2.5% to 7.0% w/w, of dihydroquercetin;
from 0.05% to 5% w/w, preferably from 0.1% to 3% w/w, more preferably from 0.5% to 2% w/w, of alpha-tocopherol;
from 0.01% to 10.0% w/w, preferably from 0.1% to 8.0% w/w, more preferably from 1% to 4% w/w, of bisabolol; and
from 0.01% to 1.0% w/w, preferably from 0.1% to 1.0% w/w, more preferably from 0.1% to 0.6% w/w, of a compound of formula (I) as previously described.

In one embodiment, the composition comprises:
from 0.1% to 10.0% w/w, preferably from 2% to 8.0% w/w, more preferably from 2.5% to 7.0% w/w, of dihydroquercetin;
from 0.05% to 5% w/w, preferably from 0.1% to 3% w/w, more preferably from 0.5% to 2% w/w, of alpha-tocopherol;
from 0.01% to 10.0% w/w, preferably from 0.1% to 8.0% w/w, more preferably from 1% to 4% w/w, of bisabolol;
from 0.01% to 1.0% w/w, preferably from 0.1% to 1.0% w/w, more preferably from 0.1% to 0.6% w/w, of a compound of formula (I) as previously described; and
   a pharmaceutically and/or cosmetically acceptable vehicle.

In one embodiment, the composition comprises:
from 0.1% to 10.0% w/w, preferably from 2% to 8.0% w/w, more preferably from 2.5% to 7.0% w/w, of dihydroquercetin;
from 0.05% to 5% w/w, preferably from 0.1% to 3% w/w, more preferably from 0.5% to 2% w/w, of alpha-tocopherol;
from 0.01% to 10.0% w/w, preferably from 0.1% to 8.0% w/w, more preferably from 1% to 4% w/w, of bisabolol; and
from 0.01% to 1.0% w/w, preferably from 0.1% to 1.0% w/w, more preferably from 0.1% to 0.6% w/w, of a compound of formula (Ia), (Ib) or (Ic) as previously described.

In one embodiment, the composition comprises:
from 0.1% to 10.0% w/w, preferably from 2% to 8.0% w/w, more preferably from 2.5% to 7.0% w/w, of dihydroquercetin;
from 0.05% to 5% w/w, preferably from 0.1% to 3% w/w, more preferably from 0.5% to 2% w/w, of alpha-tocopherol;
from 0.01% to 10.0% w/w, preferably from 0.1% to 8.0% w/w, more preferably from 1% to 4% w/w, of bisabolol;
from 0.01% to 1.0% w/w, preferably from 0.1% to 1.0% w/w, more preferably from 0.1% to 0.6% w/w, of a compound of formula (Ia), (Ib) or (Ic) as previously described; and
   a pharmaceutically and/or cosmetically acceptable vehicle.

In one embodiment, the composition comprises:
from 0.1% to 10.0% w/w, preferably from 2% to 8.0% w/w, more preferably from 2.5% to 7.0% w/w, of dihydroquercetin;
from 0.05% to 5% w/w, preferably from 0.1% to 3% w/w, more preferably from 0.5% to 2% w/w, of alpha-tocopherol;
from 0.01% to 10.0% w/w, preferably from 0.1% to 8.0% w/w, more preferably from 1% to 4% w/w, of bisabolol; and
from 0.01% to 1.0% w/w, preferably from 0.1% to 1.0% w/w, more preferably from 0.1% to 0.6% w/w, of a compound of formula (Ib) or (Ic) as previously described.

In one embodiment, the composition comprises:
from 0.1% to 10.0% w/w, preferably from 2% to 8.0% w/w, more preferably from 2.5% to 7.0% w/w, of dihydroquercetin;
from 0.05% to 5% w/w, preferably from 0.1% to 3% w/w, more preferably from 0.5% to 2% w/w, of alpha-tocopherol;
from 0.01% to 10.0% w/w, preferably from 0.1% to 8.0% w/w, more preferably from 1% to 4% w/w, of bisabolol;
from 0.01% to 1.0% w/w, preferably from 0.1% to 1.0% w/w, more preferably from 0.1% to 0.6% w/w, of a compound of formula (Ib) or (Ic) as previously described; and
   a pharmaceutically and/or cosmetically acceptable vehicle.

In one embodiment, the composition comprises:
from 0.1% to 10.0% w/w, preferably from 2% to 8.0% w/w, more preferably from 2.5% to 7.0% w/w, of dihydroquercetin;
from 0.05% to 5% w/w, preferably from 0.1% to 3% w/w, more preferably from 0.5% to 2% w/w, of alpha-tocopherol;
from 0.01% to 10.0% w/w, preferably from 0.1% to 8.0% w/w, more preferably from 1% to 4% w/w, of bisabolol; and
from 0.01% to 1.0% w/w, preferably from 0.1% to 1.0% w/w, more preferably from 0.1% to 0.6% w/w, of 3-(2-chloroacetamido) ethyl benzoate.

In one embodiment, the composition comprises:
from 0.1% to 10.0% w/w, preferably from 2% to 8.0% w/w, more preferably from 2.5% to 7.0% w/w, of dihydroquercetin;
from 0.05% to 5% w/w, preferably from 0.1% to 3% w/w, more preferably from 0.5% to 2% w/w, of alpha-tocopherol;
from 0.01% to 10.0% w/w, preferably from 0.1% to 8.0% w/w, more preferably from 1% to 4% w/w, of bisabolol;
from 0.01% to 1.0% w/w, preferably from 0.1% to 1.0% w/w, more preferably from 0.1% to 0.6% w/w, of 3-(2-chloroacetamido) ethyl benzoate; and
   a pharmaceutically and/or cosmetically acceptable vehicle.

In one embodiment, the composition comprises:
from 0.1% to 10.0% w/w, preferably from 2% to 8.0% w/w, more preferably from 2.5% to 7.0% w/w, of dihydroquercetin;
from 0.05% to 5% w/w, preferably from 0.1% to 3% w/w, more preferably from 0.5% to 2% w/w, of alpha-tocopherol;
from 0.01% to 10.0% w/w, preferably from 0.1% to 8.0% w/w, more preferably from 1% to 4% w/w, of bisabolol; and
from 0.01% to 1.0% w/w, preferably from 0.1% to 1.0% w/w, more preferably from 0.1% to 0.6% w/w, of 4-(2-chloroacetamido) ethyl benzoate.

In one embodiment, the composition comprises:
from 0.1% to 10.0% w/w, preferably from 2% to 8.0% w/w, more preferably from 2.5% to 7.0% w/w, of dihydroquercetin;
from 0.05% to 5% w/w, preferably from 0.1% to 3% w/w, more preferably from 0.5% to 2% w/w, of alpha-tocopherol;
from 0.01% to 10.0% w/w, preferably from 0.1% to 8.0% w/w, more preferably from 1% to 4% w/w, of bisabolol;
from 0.01% to 1.0% w/w, preferably from 0.1% to 1.0% w/w, more preferably from 0.1% to 0.6% w/w, of 4-(2-chloroacetamido) ethyl benzoate; and
   a pharmaceutically and/or cosmetically acceptable vehicle.

According to another aspect, the invention relates to a composition comprising a compound of formula (I), (Ia), (Ib) or (Ic) as previously described, in association with alpha-tocopherol or dihydroquercetin.

In one embodiment, the composition for use according to the present invention comprises dihydroquercetin and (3-(2-chloroacetamido) ethyl benzoate) (3-(2-chloroacetamido) ethyl benzoate).

In one embodiment, the composition for use according to the present invention comprises dihydroquercetin and (4-(2-chloroacetamido) ethyl benzoate).

In one embodiment, the composition for use according to the present invention comprises alpha-tocopherol and (3-(2-chloroacetamido) ethyl benzoate)3-(2-chloroacetamido) ethyl benzoate.

In one embodiment, the composition for use according to the present invention comprises alpha-tocopherol and (4-(2-chloroacetamido) ethyl benzoate).

In one embodiment, the composition comprises:
from 0.01% to 1.0% w/w, preferably from 0.1% to 1.0% w/w, more preferably from 0.1% to 0.6% w/w, of (3-(2-chloroacetamido) ethyl benzoate); and
from 0.05% to 5% w/w, preferably from 0.1% to 3% w/w, more preferably from 0.5% to 2% w/w, of alpha-tocopherol.

In one embodiment, the composition comprises:
from 0.01% to 1.0% w/w, preferably from 0.1% to 1.0% w/w, more preferably from 0.1% to 0.6% w/w, of (3-(2-chloroacetamido) ethyl benzoate);
from 0.05% to 5% w/w, preferably from 0.1% to 3% w/w, more preferably from 0.5% to 2% w/w, of alpha-tocopherol; and
a pharmaceutically and/or cosmetically acceptable vehicle.

In one embodiment, the composition comprises:
from 0.01% to 1.0% w/w, preferably from 0.1% to 1.0% w/w, more preferably from 0.1% to 0.6% w/w, of (3-(2-chloroacetamido) ethyl benzoate); and
from 0.1% to 10.0% w/w, preferably from 2% to 8.0% w/w, more preferably from 2.5% to 7.0% w/w, of dihydroquercetin.

In one embodiment, the composition comprises:
from 0.01% to 1.0% w/w, preferably from 0.1% to 1.0% w/w, more preferably from 0.1% to 0.6% w/w, of (3-(2-chloroacetamido) ethyl benzoate);
from 0.1% to 10.0% w/w, preferably from 2% to 8.0% w/w, more preferably from 2.5% to 7.0% w/w, of dihydroquercetin; and
   a pharmaceutically and/or cosmetically acceptable vehicle.

In one embodiment, the composition comprises:
from 0.01% to 1.0% w/w, preferably from 0.1% to 1.0% w/w, more preferably from 0.1% to 0.6% w/w, of (4-(2-chloroacetamido) ethyl benzoate); and
from 0.05% to 5% w/w, preferably from 0.1% to 3% w/w, more preferably from 0.5% to 2% w/w, of alpha-tocopherol.

In one embodiment, the composition comprises:
from 0.01% to 1.0% w/w, preferably from 0.1% to 1.0% w/w, more preferably from 0.1% to 0.6% w/w, of (4-(2-chloroacetamido) ethyl benzoate);
from 0.05% to 5% w/w, preferably from 0.1% to 3% w/w, more preferably from 0.5% to 2% w/w, of alpha-tocopherol; and
a pharmaceutically and/or cosmetically acceptable vehicle.

In one embodiment, the composition comprises:
from 0.01% to 1.0% w/w, preferably from 0.1% to 1.0% w/w, more preferably from 0.1% to 0.6% w/w, of (4-(2-chloroacetamido) ethyl benzoate); and
from 0.1% to 10.0% w/w, preferably from 2% to 8.0% w/w, more preferably from 2.5% to 7.0% w/w, of dihydroquercetin.

In one embodiment, the composition comprises:
from 0.01% to 1.0% w/w, preferably from 0.1% to 1.0% w/w, more preferably from 0.1% to 0.6% w/w, of (4-(2-chloroacetamido) ethyl benzoate);
from 0.1% to 10.0% w/w, preferably from 2% to 8.0% w/w, more preferably from 2.5% to 7.0% w/w, of dihydroquercetin; and
   a pharmaceutically and/or cosmetically acceptable vehicle.

In one embodiment, the composition further comprises at least one compound selected from triglycerides animal fat, vegetable fat, higher alcohols, glycols, mineral oil and a mixture thereof. In one embodiment, the composition comprises an acceptable vehicle which comprises at least one compound selected from animal fat, vegetable fat, higher alcohols, glycols, mineral oil and a mixture thereof. A non-limitative example of animal fat is stearic acid. Examples of vegetable fat include, but are not limited to linoleic acid, jojoba oil (also called Simmondsia chinensis oil), sweet almond oil, avocado oil, or a mixture thereof. Examples of higher alcohols include, but are not limited to cetearyl alcohol, stearyl alcohol, or cetylic alcohol. Examples of glycols include, but are not limited to propylene glycol. Examples of mineral oil include, but are not limited to paraffin oil.

In one embodiment, the composition further comprises water.

In one embodiment, the composition further comprises at least one component selected from surfactants, pigments, stabilizers, emollients, humectants, perfumes, preservatives, and mixture thereof. Examples of surfactants include, but are not limited to PEG-100 stearate, PEG-20 stearate or a mixture thereof. Examples of stabilizers include, but are not limited to carbomer. Examples of pigments include, but are not limited to zinc oxide. Examples of emollients include, but are not limited to caprylic/capric triglyceride, dicapryl ether, glyceryl stearate, glyceryl monostearate or a mixture thereof. Examples of humectants include, but are not limited to glycerin, sorbitol or a mixture thereof. Examples of perfume include, but are not limited to citronellol, geraniol, limonene, cinnamyl alcohol, butyl phenyl methylpropional, or a mixture thereof. Examples of preservatives include, but are not limited to imidazolidinyl urea.

In one embodiment, the composition further comprises an additional therapeutically active agent. Examples of therapeutically active agent include, but are not limited to, antivirals, antibiotics, antifungals, antiparasitic drugs, or monoclonal antibodies. In a preferred embodiment, the therapeutically active agent is an antiparasitic drug.

In one embodiment, the composition is designed for topical administration. In one embodiment, the composition is under a form adapted for topical administration. In one embodiment, the composition is a cream, a gel, an ointment, a solution, an emulsion, a mask, a milk, a lotion, a serum, a paste, a stick, a foam or a suspension. In a preferred embodiment, the composition is a cream or a stick. In another preferred embodiment, the composition is a gel. In a further preferred embodiment, the composition is an oil-in-water emulsion.

In an embodiment, the composition is a therapeutic composition. In one embodiment, the composition is a pharmaceutical composition. In one embodiment, the composition is a cosmetic composition.

In one embodiment, the composition is stored in a container, preferably a glass container. In an embodiment, the glass container is sterilized using a dry heat sterilizer. In an embodiment, the container is a plastic container. In an embodiment, the plastic container is sterilized using UV irradiation using low-pressure “Hard Quartz Glass” UV Lamps. In one embodiment, the composition is stable over one year in standard storage conditions.

In one embodiment, the leishmaniasis is visceral leishmaniasis (VL), cutaneous leishmaniasis (CL) or mucosal leishmaniasis (ML). In a preferred embodiment, the leishmaniasis is cutaneous leishmaniasis (CL).

In one embodiment, the parasitic protozoa responsible of leishmaniasis is Leishmania aethiopica, Leishmania amazonensis, Leishmania braziliensis, Leishmania donovani, Leishmania guyanensis, Leishmania infantum, Leishmania lainsoni, Leishmania lindenbergi, Leishmania mexicana, Leishmania major, Leishmania naiffi, Leishmania panamensis, Leishmania peruviana, Leishmania shawi, Leishmania tropica and/or Leishmania venezuelensis. In a preferred embodiment, the parasitic protozoa responsible of leishmaniasis is Leishmania amazonensis, Leishmania donovani, Leishmania mexicana and/or Leishmania major.

In one embodiment, leishmaniasis-related symptoms are related to an inflammation induced by a leishmaniasis parasite infection of the skin or the mucosa.

In one embodiment, treating leishmaniasis especially means preventing and/or reducing visible signs of leishmaniasis. The term “visible signs of leishmaniasis” includes but is not limited to skin or mucosal lesions such as papules, nodular plaques and open or closed sores.

In one embodiment, treating leishmaniasis means preventing and/or limiting cutaneous or mucosal discomfort induced by leishmaniasis. Examples of discomfort induced by leishmaniasis include, but are not limited to, itching and pain.

In one embodiment, the composition is for external use. In one embodiment, the use comprises a step of topical administration of the composition. In an embodiment, the use requires the composition to be applied on inflamed skin or mucosa.

In one embodiment, the amount of composition applied is sufficient to cover the afflicted area of the skin or mucosa with a thin layer of the composition. In one embodiment, the composition is to be rubbed into the skin or mucosa until little or no residue remains on the skin or mucosa. In one embodiment, the use requires the composition to be applied on the skin or mucosa by applying a regular massage.

In one embodiment, the composition may be applied one, two, three or more times a day. In one embodiment, the composition may be applied during 7, 14 or 21 days or until the visible symptoms of leishmaniasis disappear.

In a second aspect, the invention relates to a method of treating a subject suffering from leishmaniasis by administering an effective amount of a composition as previously described comprising dihydroquercetin (DHQ), alpha-tocopherol and optionally a compound of formula (I) and/or bisabolol, to a subject in need thereof. In one embodiment, the subject was not treated previously with another treatment for leishmaniasis. In another embodiment, the subject previously received one or more other treatments for leishmaniasis prior to be administered with the composition as described herein.

In one embodiment, the method comprises a simultaneous or sequential administration of an effective dose of least one antiparasitic drug. In one embodiment, the simultaneous or sequentially administrated drug is selected from a group comprising or consisting of meglumine antimoniate, sodium stibogluconate, miltefosine and amphotericin B. The effective dose of the least one antiparasitic drug can be determined by one skilled in the art. In one embodiment, the effective dose of the least one antiparasitic drug is at least 10%, preferably at least 20%, even more preferably at least 30% inferior to the effective dose when said antiparasitic drug is administrated as such (monotherapy).

In a third aspect, the invention relates to the use of a composition as previously described comprising dihydroquercetin (DHQ), bisabolol, and optionally alpha-tocopherol, in the manufacture of a medicament for the treatment of leishmaniasis.

In a fourth aspect, the invention relates to a device comprising a composition for use as previously described comprising dihydroquercetin alpha-tocopherol, and optionally a compound of formula (I) and/or bisabolol. In one embodiment, the device is a delivery device. In one embodiment, the device is a medical device.

In a fifth aspect, the invention relates to a kit comprising a composition for use as previously described and/or a device comprising a composition for use as previously described.

In a sixth aspect, the invention relates to a process for manufacturing a composition for use as previously described comprising dihydroquercetin (DHQ), bisabolol, and alpha-tocopherol. In an embodiment, the process of the invention comprises a step of blending DHQ, bisabolol, and alpha-tocopherol, with an acceptable vehicle, especially a pharmaceutically and/or cosmetically acceptable vehicle. In one embodiment, the process comprises a preliminary step of dissolving DHQ in jojoba oil (Simmondsia chinensis), sweet almond oil or avocado oil before blending DHQ, bisabolol, and alpha-tocopherol, with an acceptable vehicle. In one embodiment, the acceptable vehicle, especially the pharmaceutically and/or cosmetically acceptable vehicle, more especially the cosmetically acceptable base, is manufactured by any conventional method known of a person skilled in the art.

### Examples

The present invention is further illustrated by the following examples.

### Example 1: Compositions for Use According to the Invention

Compositions comprising DHQ (dihydroquercetin), bisabolol, optionally alpha-tocopherol and a cosmetically and pharmaceutically acceptable vehicle being an oil-in-water emulsion are presented in Table 1 below.

TABLE 1

| | | | Alpha- | |
|---|---|---|---|---|
| Composition | DHQ | Bisabolol | tocopherol | |
| number | (% w/w) | (% w/w) | (% w/w) | Vehicle |
| #1 | 0.5 | 0.2 | 0 | qsp 100% |
| #2 | 1 | 0.5 | 0 | qsp 100% |
| #3 | 2.5 | 1.5 | 0 | qsp 100% |
| #4 | 5.0 | 2.5 | 0 | qsp 100% |
| #5 | 7.0 | 3.0 | 0 | qsp 100% |
| #6 | 5.0 | 2.5 | 0.5 | qsp 100% |
| #7 | 7.0 | 3.0 | 1.0 | qsp 100% |

The oil-in-water emulsion used as vehicle in the compositions of Table 1 has the following composition:

AQUA;

PRUNUS AMYGDALUS DULCIS OIL;

CAPRYLIC/CAPRIC TRIGLYCERIDE;

OLUS OIL;

BUTYROSPERMUM PARKII BUTTER;

ALCOHOL;

CETEARYL ALCOHOL;

DICAPRYLYL ETHER;

GLYCERIN;

PROPANEDIOL;

SORBITOL;

GLYCERYL STEARATE;

GLYCERYL STEARATE CITRATE;

POLYGLYCERYL-3 METHYLGLUCOSE DISTEARATE;

XANTHAN GUM;

SODIUM DEHYDROACETATE;

SODIUM BENZOATE;

PHENOXYETHANOL; and

CITRIC ACID.

### Example 2: In Vitro Anti-Leishmanial Activity

dihydroquercetin, 3-(2-chloroacetamido) ethyl and bisabolol were assessed for their anti-leishmanial activity.

The direct microbicidal effect of compounds against the Leishmania parasites in infected macrophages and neutrophils was evaluated.

Bone marrow-derived macrophages (BMDM) were cultured for 7 days, harvested and infected with Leishmania major mCherry for 2 hrs. Then the non-phagocytosed parasites were washed and dihydroquercetin, 3-(2-chloroacetamido) ethyl benzoate or bisabolol were added at different concentrations and incubated at different time points.
Parasites killing was evaluated by flow cytometry and microscopy.
NO production was assessed by Griess reaction from the supernatants.

Neutrophils were isolated by Magnetic-activated cell sorting (MACS) and infected with L. major mCherry at a multiplicity of infection ratio (MOI) of 1:10.

Dihydroquercetin, 33-(2-chloroacetamido) ethyl benzoate or bisabolol at different concentrations were added and stimulated for 24 hours.

Killing of the parasites was measured by flow cytometry.

Kinetic of ROS production was measured by Luminol for 90 min.

As expected, 3-(2-chloroacetamido) ethyl benzoate had a direct microbicidal effect against L. major. Furthermore, 3-(2-chloroacetamido) ethyl benzoate induced a decrease in ROS production.

Dihydroquercetin and bisabolol individually were found to have no impact on macrophage mediated parasite killing.

### Example 3: In Vitro Toxicity Evaluation of Separate Components

Neutrophil cell viability was assessed by flow cytometry with DAPI staining pas 24 hours incubation with dihydroquercetin (5, 15 and 25 μM), 3-(2-chloroacetamido) ethyl benzoate (1, 2 and 4 μM) and bisabolol (0.05, 0.1 and 0.5% w/v).

Dihydroquercetin and bisabolol showed no cytotoxicity.

However, 3-(2-chloroacetamido) ethyl benzoate in the assessed concentrations induced a reduction in neutrophil viability.

### Example 4: In Vivo Anti-Leishmanial Activity

The anti-leishmanial activity of the following compositions was assessed:
A. dihydroquercetin (3%, 5%, 7% w/w);
B. bisabolol (1%, 3%; 5% w/w);
C. 3-(2-chloroacetamido) ethyl benzoate (0.1%, 0.5%, 1% w/w);
D. dihydroquercetin/alpha-tocopherol (5%/0.5% respectively);
E. dihydroquercetin/alpha-tocopherol/bisabolol (5%/0.5%/1-5% respectively);
F. 3-(2-chloroacetamido) ethyl benzoate/alpha-tocopherol (0.5% w/w each);
G. 3-(2-chloroacetamido) ethyl benzoate/dihydroquercetin/alpha-tocopherol (0.5%/5%/0.5% w/w respectively);
H. 3-(2-chloroacetamido) ethyl benzoate/dihydroquercetin/alpha-tocopherol/bisabolol (0.5%/5%/0.5%/1-5% w/w respectively);

BALB/c mice were infected with 1×105 metacyclic promastigotes of L. major in the ear dermis. Once the lesion reaches a score of 2 (signs of inflammation):
10 μl of the test compositions A-G formulated as topical cream were locally administrated daily with an insulin syringe without needle covering the lesion.
Topical cream vehicle was administered in the same way as a control.

At the first signals of necrosis the mice were euthanized and the immune response analyzed by Flow cytometry. Parasite Load was analyzed by LDA (limitant dilution assay).

The treatment of the L. major and L. mexicana infected mice with test composition C 3-(2-chloroacetamido) ethyl benzoate 0.1% showed a significant inhibition of the lesion development.

Surprisingly, the treatment of the L. major and L. mexicana infected mice with the test composition D (dihydroquercetin/alpha-tocopherol 5%/0.5% respectively) yielded a similar lesion inhibition as the 3-(2-chloroacetamido) ethyl benzoate 0.1% treatment.

Furthermore, the association of 3-(2-chloroacetamido) ethyl benzoate at 0.5% with dihydroquercetin and/or alpha-tocopherol yielded similar inhibition of the lesion development as the test composition of 3-(2-chloroacetamido) ethyl benzoate at a concentration of 1% w/w. Therefore, the synergy among 3-(2-chloroacetamido) ethyl benzoate with dihydroquercetin and/or alpha-tocopherol results to the same beneficial biological effects while limiting the effective dose of 3-(2-chloroacetamido) ethyl benzoate.

1. A composition comprising dihydroquercetin and alpha-tocopherol for use in the treatment of leishmaniasis.

2. The composition for use according to claim 1, wherein the concentration of dihydroquercetin in the composition ranges from 2% to 8.0% w/w, preferably from 2.5% to 7.0% w/w; and wherein the concentration of alpha-tocopherol in the composition ranges from 0.1% to 3% w/w, preferably from 0.5% to 2% w/w, in weight to the total weight of the composition.

3. The composition for use according to claim 1, further comprising bisabolol.

4. The composition for use according to claim 3, wherein the concentration of bisabolol in the composition ranges from 0.1% to 8.0% w/w, preferably from 2.5% to 4.0% w/w, in weight to the total weight of the composition.

5. The composition for use according to claim 1, further comprising a compound of formula (I): 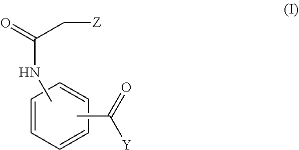
wherein: Z represents a halogen atom selected from the group consisting of Cl, Br, I and F; and Y represents a substituent selected from a group consisting of C₂H₅O—, —NH—CO—NH—R in which R represents H, an alkyl or aryl group and N—R1 wherein R1 is an alkyl or heterocyclic group.

6. The composition for use according to claim 5, wherein the compound of formula (I) is 3-(2-chloroacetamido) ethyl benzoate or 4-(2-chloroacetamido) ethyl benzoate.

7. The composition for use according to claim 5, wherein the concentration of the compound of formula (I) in the composition ranges from 0.05% to 1.0% w/w, preferably from 0.1% to 0.6% w/w, in weight to the total weight of the composition.

8. The composition for use according to claim 1, further comprising at least one pharmaceutically and/or cosmetically acceptable vehicle.

9. The composition for use according to claim 8, wherein said at least at least one pharmaceutically and/or cosmetically acceptable vehicle is selected from triglycerides, animal fat, vegetable fat, higher alcohols, glycols, mineral oil, and a mixture thereof.

10. The composition for use according to claim 1, wherein said composition further comprises at least one component selected from surfactants, pigments, stabilizers, emollients, humectants, perfumes, preservatives, and a mixture thereof.

11. The composition for use according to claim 1, wherein said composition is a cream, a gel, an ointment, a solution, an emulsion, a mask, a milk, a lotion, a serum, a paste, a stick, a foam or a suspension; preferably a cream or a stick.

12. The composition for use according to claim 1, wherein the leishmaniasis is visceral leishmaniasis, cutaneous leishmaniasis or mucosal leishmaniasis, preferably the leishmaniasis is cutaneous leishmaniasis.

13. The composition for use according to claim 1, wherein the parasitic protozoa responsible of leishmaniasis is Leishmania aethiopica, Leishmania amazonensis, Leishmania braziliensis, Leishmania donovani, Leishmania guyanensis, Leishmania infantum, Leishmania lainsoni, Leishmania lindenbergi, Leishmania mexicana, Leishmania major, Leishmania naiffi, Leishmania panamensis, Leishmania peruviana, Leishmania shawi, Leishmania tropica and/or Leishmania venezuelensis; preferably Leishmania amazonensis, Leishmania donovani, Leishmania mexicana and/or Leishmania major.

14. A pharmaceutical and/or cosmetic composition for use in the treatment of leishmaniasis, said pharmaceutical and/or cosmetic composition comprising a composition as described in claim 1.

15. A device, preferably a delivery device, for use in the treatment of leishmaniasis, said device comprising a composition as described in claim 1.

16. The composition for use according to claim 2, further comprising bisabolol.

17. The composition for use according to claim 2, further comprising a compound of formula (I): 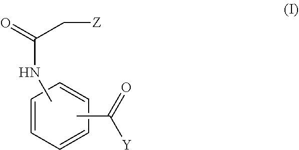
wherein: Z represents a halogen atom selected from the group consisting of Cl, Br, I and F; and Y represents a substituent selected from a group consisting of C₂H₅O—, —NH—CO—NH—R in which R represents H, an alkyl or aryl group and N—R1 wherein R1 is an alkyl or heterocyclic group.

18. The composition for use according to claim 3, further comprising a compound of formula (I): 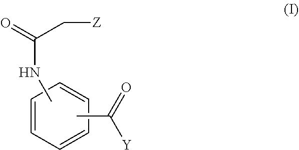
wherein: Z represents a halogen atom selected from the group consisting of Cl, Br, I and F; and Y represents a substituent selected from a group consisting of C₂H₅O—, —NH—CO—NH—R in which R represents H, an alkyl or aryl group and N—R1 wherein R1 is an alkyl or heterocyclic group.

19. The composition for use according to claim 4, further comprising a compound of formula (I): 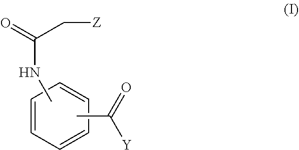
wherein: Z represents a halogen atom selected from the group consisting of Cl, Br, I and F; and Y represents a substituent selected from a group consisting of C₂H₅O—, —NH—CO—NH—R in which R represents H, an alkyl or aryl group and N—R1 wherein R1 is an alkyl or heterocyclic group.

20. The composition for use according to claim 6, wherein the concentration of the compound of formula (I) in the composition ranges from 0.05% to 1.0% w/w, preferably from 0.1% to 0.6% w/w, in weight to the total weight of the composition.
